(19) United States
(12) Patent Application Publication
(10) Pub. No.: US 2007/0231403 A1
(43) Pub. Date: Oct. 4, 2007

(54) Natural composite preservatives

(76) Inventors: Deok Hoon Park, Yongin-si (KR); Jong Sung Lee, Anyang-si (KR); Seong Taek Hong, Nam-gu (KR); Chang Gu Hyun, Jeju-si (KR); Sang Yeb Lee, Seoul (KR)

Correspondence Address: O'KEEFE, EGAN, PETERMAN & ENDERS LLP, 1101 CAPITAL OF TEXAS HIGHWAY SOUTH, #C200, AUSTIN, TX, 78746, US

(21) Appl. No.: 11/732,455

(22) Filed: Apr. 3, 2007

(30) Foreign Application Priority Data
Apr. 4, 2006 (KR) 10-2006-0030619

(51) Int. Cl. A61K 35/64; A61K 36/82; A61K 36/20

(52) U.S. Cl. 424/539; 424/729; 424/769; 424/771; 424/736

(57) ABSTRACT

Disclosed herein is a natural composite preservative comprising extracts from Camellia sinensis, Matricaria recutita, Citrus grandis, Magnolia biondii, propolis, Thujopsis dolabrata and Salix alba, in combination. The natural composite preservative has potent antibacterial activity against a wide spectrum of microorganisms in addition to being safe for the body, producing neither toxicity nor irritation, and thus can be applied to cosmetics, pharmaceuticals, and foods.

### BACKGROUND OF THE INVENTION

1. Field of the Invention

The present invention relates, in general, to a natural composite preservative and, more particularly, to a natural composite preservative prepared from a combination of extracts from Camellia sinensis, Matricaria recutita, Citrus grandis, Magnolia biondii, propolis, Thujopsis dolabrata and Salix alba. Also, the present invention relates to a product containing the same.

2. Description of the Related Art

In a great variety of articles and products, which are widely and extensively used in daily life, such as foods, pharmaceuticals, cosmetics, paints, biological samples, etc., preservatives, which are generally not beneficial to the body, must be used in order to retard spoilage. The preservatives may be natural materials, but are, for the most part, made from synthetic chemicals. Even parabens, which are known to be the safest for the body and are generally used in cosmetics and pharmaceuticals, are now suspected of acting as dermal allergens (Andrea Counti et al., Contact Dermatitis, 1997, 37; 35-36) and environmental hormones (Edwin et al., Toxicology and applied pharmacology, 1998, 153; 12-19) in addition to stimulating the development of resistant bacteria.

Taken as an example, most cosmetics provide an environment suitable for the growth of microorganisms and are apt to spoil due to chemical change, and in order to prevent this, synthetic preservatives, such as parabens, imidazolidinyl urea, diazolidinyl urea and phenoxy ethanol, are added thereto thanks to the low cost thereof. With the presentation of the dangerous problems with synthetic preservatives, many enterprises feel a need to use natural preservatives, but do not do so in practice because of the high cost and the lack of technology.

As for natural preservative materials, in addition, most of them have problems which make the commercialization thereof impossible, such as poor color and smell, low stability, narrow antibacterial spectrum, difficulty in the formulation thereof, etc. Indeed, only a few materials are commercialized now, such as hinokitiol, magnolol and DF-100, which are extracts from Thujopsis dolabrata, Magnolia biondii and Citrus grandis, respectively.

Therefore, there is the need for a natural preservative that has potential inhibitory activity against a broad spectrum of bacteria and can be provided in a stable formulation while overcoming the problems of conventional synthetic preservatives.

Leading to the present invention, intensive and thorough research into natural preservatives, conducted by the present inventors, resulted in the finding that when combined together in specific ratios, antibiotic plant extracts show collectively potent inhibitory activity against a broad spectrum of microorganisms and are safe for the body.

### SUMMARY OF THE INVENTION

Accordingly, the present invention has been made keeping in mind the above problems occurring in the prior art, and an object of the present invention is to provide a natural composite preservative which has potent inhibitory activity against a broad spectrum of microorganisms and is safe enough to be applicable to cosmetics, pharmaceuticals and foods.

It is another object of the present invention to provide a product containing the natural composite preservative.

In order to accomplish the above objects, there is provided a natural composite preservative comprising a combination of extracts from Camellia sinensis, Matricaria recutita, Citrus grandis, Magnolia biondii, propolis, Thujopsis dolabrata and Salix alba.

Also, there is provided a product comprising the natural composite preservative.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a photograph showing an agar diffusion assay for determining the antibacterial activity of the natural composite preservative against microorganisms;

FIG. 2 is a photograph showing a serial dilution test for determining the minimum inhibitory concentration (MIC) of the natural composite preservative according to the present invention;

FIG. 3 is a photograph showing the preservative effect of the natural composite preservative on cosmetics 3 days after treatment; and

FIGS. 4a and 4b are plots showing the preservative effects of the natural composite preservatives according to time (Lotion C: lotion lacking the natural composite preservative, Lotion N: containing 2% of the natural composite preservative, Toner C: lacking the natural composite preservative, Toner N: containing 2% of the natural composite preservative).

### DESCRIPTION OF THE PREFERRED EMBODIMENTS

In accordance with an aspect thereof, the present invention pertains to a natural composite preservative comprising a combination of extracts from Camellia sinensis, Matricaria recutita, Citrus grandis, Magnolia biondii, propolis, Thujopsis dolabrata and Salix alba.

The term “natural composite preservative”, as used herein, is intended to refer to a combination of two or more extracts from natural substances, which have synergistic preservative effects. Natural composite preservatives are also called naturotics. For the purpose thereof, the natural composite preservative of the present invention consists essentially of extracts from Camellia sinensis, Matricaria recutita, Citrus grandis, Magnolia biondii, propolis, Thujopsis dolabrata and Salix alba.

A detailed description will be given of each of the extracts indispensable for the natural composite preservatives of the present invention, below.

Camellia sinensis is rich in polyphenols. Of them, EGCG is known to have potent antibacterial activity, antioxidant activity, and anti-inflammatory activity. Leaves and roots thereof are used for extraction.

Chamomile (Matricaria recutita) is a plant belonging to the China aster. Containing chamazulene, bisabolol and apigenin therein, an extract from chamomile (Matricaria recutita), a composite plant, is found to have anti-inflammatory activity, a wound-healing effect and a skin-soothing effect.

Citrus grandis is a grapefruit plant belonging to the citrus genus. Its fruits are known to have various physiological and medicinal effects, including the treatment of acne and oily skin, promotion of hair growth, improvement in skin and tissue texture, cellulite removal, recovery from muscle fatigue, and anti-obesity activity.

Magnolia biondii is a tall deciduous tree belonging to the magnolia family. When dried, its flower bud is called magnoliae flos, which is known to show therapeutic effects on nasal congestion, cold, rigor, fever and general fatigue. Also, magnoliae flos has been reported to be useful in the treatment of asthma and thromboembolism.

Propolis, also known as bee glue, is a resinous substance collected by honey bees, mainly from flower buds of balsam trees and other trees. It results from the enzymatic treatment of excreta from buds and bees. Propolis is reported to have therapeutic activities on a wide range of diseases, including cancer, diabetes, inflammatory allergies (bronchial asthma, drug allergies), arteriosclerosis, hypertension, prostatitis, hypothyroid dysfunction, gynecological diseases, rheumatism, cerebrovascular insufficiency, rectal diseases, dermal diseases, etc.

Thujopsis dolabrata is an evergreen coniferous tall tree belonging to the family Cupressaceae. Its wood is suitable for construction, civil engineering, lacquered wares, ships, etc.

Salix alba is a scrub or tall tree belonging to the family Salicaceae, and an extract from the bark thereof is used as an astringent, an antipyretic, and a diuretic.

The extracts of the natural composite preservatives according to the present invention may be obtained by extracting respective raw materials in solvents, such as water, organic solvents, or combinations thereof. Extraction may be carried out individually or collectively with the raw materials. Preferably, the raw materials are individually used under respective optimal extraction conditions.

Examples of the organic solvent useful in the extraction of the present invention include alcohols, such as methanol, ethanol, isopropanol, butanol and the like, ethylene, acetone, ethers, hexane, chloroform, ethyl acetate, DMF (N,N-dimethylformamide), DMSO (dimethylsulfoxide), and combinations thereof. Typically, extraction is performed at room temperature or under warm conditions. Preferable solvents are water and ethanol. In detail, ethanol is preferably used as the extraction solvent for Camellia sinensis, Citrus grandis, Magnolia biondii, Thujopsis dolabrata, and Salix alba. The extraction of Matricaria recutita and propolis is conducted in water. The extracts can be prepared by pulverizing dried or fresh raw materials, immersing the pulverized materials in 10 to 100 volumes of respective solvents, and removing the precipitates through filtration or centrifugation. The supernatants are combined together to provide the extract.

For thermal extraction, the raw materials are added to water/organic solvent and heated to 70° C. or higher, preferably 80° C. or higher, and more preferably 90° C. or higher for 1 hr or longer, preferably 2 hrs or longer, and more preferably 4 hrs or longer, followed by filtration. It is important to correctly set the heating temperature and time period. For example, higher temperatures allow for shorter time periods of heating, but may result in destroying effective ingredients. On the other hand, at a lower temperature, effective ingredients are insufficiently extracted from the raw materials.

The extracts thus obtained, whether through dermal or cold extraction, are filtered through, for example, a nylon membrane or through cold filtration, and the filtrates may be used without further processing, or after being dried using a lyophilization method, a vacuum method, a hot air method, or a spraying method.

In the natural composite preservative according to the present invention, extracts from Citrus grandis, Camellia sinensis, propolis, Magnolia biondii, Matricaria recutita, Salix alba, and Thujopsis dolabrata are contained in a concentration ratio of 5:1 to 10:1:5:1 to 10:1:5, preferably in a ratio of 5:1 to 5:1:5:1 to 5:1:5, and more preferably in a ratio of 5:3:1:5:3:1:5.

Each of the extracts may be present in an amount of 1˜30% by weight based on the total weight of the natural composite preservative of the present invention, preferably in an amount of 1˜20% by weight, and more preferably in an amount of 1˜5% by weight.

Qualitative analysis allowed the identification of individual ingredients as EGCG, alpha-bisabolol, 2-methoxycinnamaldehyde, magnolol, caffeic acid, alpha-thujaplicin and salicin. Quantitatively, these ingredients are individually contained in concentration ratios of 0.01˜5.0, and preferably in a ratio of 0.01˜1.0.

The aforementioned individual ingredients are described only because they are major components in the natural composite preservative of the present invention. In fact, because it is in the form of an extract, the natural composite preservative of the present invention contains other additional ingredients which may play an important role in preservative activity. Therefore, the materials and ingredients useful in the present invention are not limited to those described above. For example, caffeic acid is detected in various plants such as rosemary and Brunnella vulgaris L. sub sp. asiatica Hara (B. asiatica Nakai). Thus, extracts from other materials may be used in the present invention if they are similar in ingredient and content to those described above.

The natural composite preservative of the present invention is found to have potent inhibitory activities against a broad spectrum of microorganisms, especially bacteria, fungi and yeasts (Tables 6 and 7), show antioxidant effects (Table 8), and be safe for human skin, exhibiting neither cytotoxicity (Table 9) nor acute oral toxicity.

As long as they do not inhibit the preservative activity, other additional ingredients may be included in the natural composite preservative of the present invention.

With a broad preservative spectrum, the natural composite preservative of the present invention can be applied to a great variety of articles and products such as pharmaceuticals, cosmetics, foods, fibers, paints, necessaries of life, etc. in order to preserve them for a long period of time. Thus, the natural composite preservatives of the present invention ensure the stability and safety of the products applied therewith. In addition, the natural composite preservatives of the present invention show excellent preservative activity without the concomitant production of the side effects of conventional chemical preservatives.

In accordance with another aspect thereof, therefore, the present invention pertains to a product comprising the natural composite preservative of the present invention.

Examples of the product to which the natural composite preservatives of the present invention are applicable include cosmetics, pharmaceuticals, foods, fibers, necessaries of life, and the like. The natural composite preservatives of the present invention are preferably used in cosmetics and pharmaceuticals, and more preferably in cosmetics.

When applied to a product, the natural composite preservatives of the present invention is used in an amount of 0.001˜30% by weight based on the total weight of the product, preferably in an amount of 0.001˜20% by weight, and more preferably in an amount of 0.001˜5% by weight.

For the production of the product containing the natural composite preservative of the present invention, appropriate formulations and additives may be used in a manner well known to those skilled in the art.

For example, the natural composite preservatives of the present invention may be used in various cosmetics, such as creams, lotions, skin lotions, and the like, and various life necessities, such as shampoos, cleansing agents, facial soaps, hair treatments, hair solutions, etc., so as to produce cosmetics or necessaries which are preserved for a long period of time while avoiding the problems with synthetic preservatives.

Cosmetics to which the natural composite preservatives of the present invention are applicable contain water-soluble vitamins, lipid-soluble vitamins, large molecular weight peptides, large moleculare weight polysaccharides, sphingolipids, etc.

As long as it can be formulated into cosmetics, any water-soluble vitamin can be used. Preferable are vitamin B1, vitamin B2, vitamin B6, pyridoxine, pyridoxine chloride, vitamin B12, pantothenic acid, nicotinic acid, nicotinamide, folic acid, vitamin C, and vitamin H, and salts thereof (thiamine chloride, sodium ascorbate, etc.) and derivatives thereof (ascorbic acid-2-phosphate sodium, ascorbic acid-2-phosphate magnesium, etc.) also fall within the scope of the present invention.

Any lipid-soluble vitamin, as long as it can be formulated into cosmetics, can be used in the present invention. Preferable are vitamin A, carotene, vitamin D2, vitamin D3, and vitamin E (d1-α-tocopherol, d-α-tocopherol, d-δ-tocopherol), and salts thereof (palmitic ascorbate, stearic ascorbate, acetic acid dl-α-tocopherol, nicotinic acid dl-α-tocopherol vitamin E, DL-pantothenyl alcohol, D-pantothenyl alcohol, pantothenyl ethyl ether, etc.) are also within the range of the lipid-soluble vitamins useful in the present invention.

Large molecular weight peptides can be used, as long as they are permissible for formulation into cosmetics. Examples of useful large molecular weight peptides include collagen, hydrolyzed collagen, gelatin, elastin, hydrolyzed elastin, keratins, etc.

As long as it is acceptable for formulation into cosmetics, any large molecular weight polysaccharide may be used in the present invention. Preferable are hydroxyethylcellulose, xanthan gum, sodium hyaluronate, chondroitin sulfate, and salts thereof (sodium salt). For example, chondroitin sulfate or salts thereof, isolated from mammals or fish, may be used.

As long as it is permissible for formulation into cosmetics, any sphingolipid may be used in the present invention. Preferable are ceramides, phytosphingosine, and sphingoglycolipids.

The cosmetics of the present invention may comprise other conventional components in addition to the aforementioned components.

Additional components that may be used in the present invention may be exemplified by lipid components, moisturizers, emollients, surfactants, organic and inorganic pigments, organic powders, UV absorbers, antioxidants, plant extracts, pH adjusters, alcohols, pigments, perfumes, etc.

Examples of the lipid components include ester lipids, hydrocarbon lipids, silicon-based oil, fluorine lipids, animal oil and vegetable oil, etc.

As the moisturizers, water-soluble low-molecular weight moisturizers, lipid-soluble low-molecular weight moisturizers, water-soluble large-molecular weight moisturizers, and lipid-soluble large-molecular weight moisturizers may be used.

As for the emollients, they may be exemplified by long chain acylglutamic acid cholesteryl ester, hydroxystearic acid cholesteryl ester, 12-hydroxystearic acid, stearic acid, rosin acid, and lanolic acid fatty acid cholesterylester.

The surfactants useful in the present invention may be non-ionic, anionic, cationic, or amphipathic.

Examples of the non-ionic surfactants include self-emulsifying monostearic acid glycerin, propyleneglycol fatty acid ester, glycerin fatty acid ester, polyglycerin fatty acid ester, sorbitan fatty acid ester, POE(polyoxyethyelene)sorbitan fatty acid ester, POE sorbitol fatty acid ester, POE glycerin fatty acid ester, POE alkylether, POE fatty acid ester, POE hydrogenated castor oil, POE castor oil, POE-O-POP (polyoxyethylene-O-polyoxypropylene) copolymer, POE-O-POP alkylether, polyether modified silicon, lauric acid alkanolamide, alkylamine oxide, hydrogenated bean phospholipids, etc.

Useful as the anionic surfactants are fatty acid soap, α-acylsulfonate, alkylsulfonate, alkylallyl sulfonate, alkylnaphthalene sulfonate, alkyl sulfonate, POE alkylether sulfonate, alkylamide sulfonate, alkyl phosphate, POE alkyl phosphate, alkylamide phosphate, alkyloylalkyltaurinate, N-acylamino acid salts, POE alkylether carboxylate, alkylsulfosuccinate, alkylsulfoacetate sodium, acylated hydrolyzed collagen peptide salts, and perfluoroalkylphosphoric acid ester.

Useful cationic surfactants in the present invention may be exemplified by alkyltrimethyl ammonium chloride, stearyl trimethyl ammonium chloride, stearyl trimethyl ammonium bromide, cetostearyltrimethyl ammonium chloride, distearyldimethyl ammonium chloride, stearyldimethylbenzyl ammonium chloride, behenyltrimethyl ammonium bromide, benzalkonium chloride, stearic acid diethylaminoethyl amide, stearic acid dimethylaminopropyl amide, lanolin derivatives, and tertiary ammonium salts.

As for amphipathic surfactants, they may be in the form of carboxybetain type, amidobetain type, sulfobetain type, hydroxysulfobetain, amidosulfobetain type, phosphobetain type, aminocarboxylate type, imidazolin derivative type, or amidoamine type.

Examples of pigments useful in the present invention include inorganic pigments, such as silicate, anhydrous silicate, magnesium silicate, talc, sericite, mica, kaolin, bengala, clay, bentonite, titanium-coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine blue, chrome oxide, chrome hydroxide, calamine, carbon black, and combinations thereof; organic pigments, such as polyamide, polyester, polypropylene, polystyrene, polyurethane, vinyl resin, urea resin, phenol resin, fluorine resin, silicon resin, acryl resin, melamine resin, epoxy resin, polycarbonate resin, divinylbenzene-styrene copolymer, silk powder, cellulose, CI pigment yellow, and CI pigment orange; and composites of the inorganic and the organic pigments.

The organic powders may be made of metal soap, such as calcium stearate; alkyl phosphate, such as sodium zinc cetyl phosphate, zinc lauryl phosphate, calcium lauryl phosphate, etc.; multivalent metal salts of acylamino acid, such as N-lauroyl-β-alamine calcium, N-lauroyl-β-alanine zinc, N-lauroylglycin calcium, etc.; multivalent metal salts of amidosulfonic acid, such as N-lauroyl-taurine calcium, N-palmitoyl-taurine calcium, etc; N-acyl basic amino acid, such as Nε-lauroyl-L-lysine, Nε-palmitoyl lysine, Nα-palmitoyl ornitine, Nα-lauroyl arginine, Nα-hydrogenated tallow fatty acid acyl arginine, etc.; N-acylpolypeptide, such as N-lauroylglycyl glycine; α-amino fatty acid, such as α-aminocaproic acid, α-aminolauric acid, etc.; or polyethylene, polypropylene, nylon, polymethylmethacrylate, polystyrene, divinylbenzene-styrene copolymer, tetrafluoroethylene, etc.

Examples of UV absorbers include para-aminobenzoic acid, para-aminobenzoic acid ethyl, para-aminobenzoic acid amyl, para-aminobenzoic acid octyl, salicylic acid ethylene glycol, salicylic acid phenyl, salicylic acid octyl, salicylic acid benzyl, salicylic acid butylphenyl, salicylic acid homomenthyl, cinnamic acid benzyl, para-methoxy cinnamic acid 2-ethoxyethyl, para-methoxycinnamic acid octyl, dipara-methoxycinnamic acid mono2-ethylhexane glyceryl, para-methoxycinnamic acid isopropyl, diisopropyl-diisopropyl cinnamic acid ester mixture, urocanic acid, urocanic acid ethyl, hydroxymethoxybenzophenone, hydroxymethoxybenzophenone sulfonic acid and salts thereof, dihydroxymethoxybenzophenone, sodium dihydroxymethoxybenzophenone disulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl)benzotriazole, etc.

Alcohols useful in the present invention are higher alcohols, such as cetyl alcohol.

The cosmetics of the present invention may be in the form of face wash cream, face wash foam, cleansing cream, cleansing milk, cleansing lotion, massage cream, cold cream, moisturizing cream, emollient, astringent, pack, after-shaving cream, sun block cream, suntan oils, soaps, body shampoo, hair shampoo, hair rinse, hair treatment, hair growth tonic, hair cream, hair liquid, set lotion, hair spray, hair bridge, color rinse, color spray, permanent wave liquid, press powder, loose powder, eye shadow, hand cream, lipstick, etc.

A better understanding of the present invention may be obtained through the following examples, which are set forth to illustrate, but are not to be construed as the limit of the present invention.

### EXAMPLE 1

### Preparation of Extracts

Vegetable extracts useful in the natural composite preservatives of the present invention were prepared as follows.

One kg of leaves and/or roots of Camellia sinensis was finely cut using a grinder and added to 100 kg of 70% ethanol, followed by refluxing at 100° C. for 4 hrs with a reflux apparatus. The extract thus formed was filtered using a filter aid and incubated in a cold place for 7˜10 days, after which the precipitates were removed. The resulting extract was mixed with additional 70% ethanol to achieve a total amount of 100 kg.

One kg of leaves and/or roots of chamomile (Matricaria recutita) was finely cut using a grinder and added to 100 kg of purified water before heating extraction at 100° C. for 4 hrs. The extract thus obtained was filtered using a filtration aid, and the filtrate was left in a cold place for 7˜10 days. After removal of the precipitates, the extract was mixed with purified water to achieve a total amount of 100 kg.

One kg of fruit of Citrus grandis was finely cut using a grinder and added to 100 kg of 10% ethanol, followed by heating extraction at 100° C. for 4 hrs. The extract was filtered using a filtration aid and mixed with 10% ethanol to achieve a total amount of 100 kg.

One kg of bark of Magnolia biondii was finely cut and added to 100 kg of 10% ethanol, followed by heating extraction at 100° C. for 4 hrs. The filtrate was left in a cold place for 7˜10 days. After removal of the precipitates, the extract was mixed with 10% ethanol to achieve a total amount of 100 kg.

One kg of propolis was finely cut and added to 100 kg of purified water, followed by heating extraction at 100° C. for 1 hr. The extract was filtered and left in a cold place for 7˜10 days. After removal of the precipitates, the extract was mixed with purified water to achieve a total amount of 100 kg.

One kg of bark of Thujopsis dolabrata was dried, finely pulverized, and added to 10 volumes of ethanol, followed by extraction at room temperature for 24 hrs. The extract was filtered, and the filtrate was concentrated in a vacuum to afford a powder.

One kg of bark of Salix alba was finely cut using a grinder and added to 100 kg of 10% ethanol, followed by heating extraction at 100° C. for 4 hrs. The extract was filtered using a filtration aid and mixed with additional 10% ethanol to achieve a total amount of 100 kg.

### EXAMPLE 2

### Antibacterial Activity of Extract

Natural extracts, known to have antibacterial activity, were screened for inhibitory activity against Staphylococcus aureus and Escherichia coli. For this antibacterial assay, an agar diffusion method was employed, in which disks coated with a known concentration of antibiotics were placed on bacteria-coated plates and incubated to generate growth inhibition halos around the antibiotic disks. The diameters of the growth inhibition halos depended on the concentrations of the antibiotics applied to the disks. The Kirby-Bauer method is currently widely used as an agar diffusion method. The extracts were found to have high antibacterial activity as assayed by this method. The concentration used in the antibacterial assay was limited to 100 ppm, and the results are summarized in Table 1, below.

TABLE 1
Assay of Natural Extracts for Antibacterial Activity

Inhibitory Activity Against

| | Staphylococcus | Escherichia |
|---|---|---|
| Extract Sources | aureus | coli |
| Camellia sinensis | ++ | ++ |
| Matricaria recutita | ++ | ++ |
| Citrus grandis | +++ | ++ |
| Magnolia biondii | +++ | +++ |
| Propolis | ++ | ++ |
| Thujopsis dolabrata | +++ | +++ |
| Salix alba | ++ | ++ |

+: halo diameter not more than 3 mm
++: halo diameter from 4 to 10 mm
+++: halo diameter from 11 to 13 mm
++++: halo diameter from 14 to 16 mm
+++++: halo diameter not less than 17 mm

### EXAMPLE 3

### Preparation of Naturotic

Of the natural extracts assayed for antibacterial activity against Staphylococcus aureus and Escherichia coli in Example 2, extracts from Camellia sinensis, Matricaria recutita, Citrus grandis, Magnolia biondii, propolis, Thujopsis dolabrata and Salix alba were combined in various concentration ratios to afford natural composite preservatives, the final concentration of which was set at 100 ppm (Table 2). The natural composite preservatives were assayed for antibacterial activity against Staphylococcus aureus using an agar diffusion method. The results are given in Table 3. As is apparent from the data of Table 1 for individual extracts and Table 3 for composite extracts, the natural composite preservatives were far superior in antibacterial activity to individual extracts, showing synergistic effects attributable to the combination of individual extracts with the antibacterial activity peaking at a concentration ratio of 5:3:1:5:3:1:5 Citrus grandis:Camellia sinensis:propolis:Magnolia biondii:Matricaria recutita:Salix alba:Thujopsis dolabrata, as in combination 23. These results show that although the individual extracts have antibacterial activity, none of the combinations thereof have the same antibacterial activity or improved synergistic effects. On the contrary, the antibacterial activity is reduced in some combinations. In consideration of the difficulty in finding particular extract combinations which have synergistic effects, the present invention cannot be simply derived from conventional techniques.

TABLE 2
Concentration Combinations of Individual Extracts in Natural
composite preservatives with a Final Concentration of 100 ppm

| | Citrus | Camellia | | Magnolia | | Salix | Thujopsis |
|---|---|---|---|---|---|---|---|
| No. | grandis | sinensis | propolis | biondii | Matricaria recutita | alba | dolabrata |
| 1 | 5 | 1 | 1 | 5 | 1 | 1 | 5 |
| 2 | 5 | 2 | 1 | 5 | 1 | 1 | 5 |
| 3 | 5 | 3 | 1 | 5 | 1 | 1 | 5 |
| 4 | 5 | 4 | 1 | 5 | 1 | 1 | 5 |
| 5 | 5 | 5 | 1 | 5 | 1 | 1 | 5 |
| 6 | 5 | 1 | 2 | 5 | 1 | 1 | 5 |
| 7 | 5 | 1 | 3 | 5 | 1 | 1 | 5 |
| 8 | 5 | 1 | 4 | 5 | 1 | 1 | 5 |
| 9 | 5 | 1 | 5 | 5 | 1 | 1 | 5 |
| 10 | 5 | 1 | 1 | 5 | 2 | 1 | 5 |
| 11 | 5 | 1 | 1 | 5 | 3 | 1 | 5 |
| 12 | 5 | 1 | 1 | 5 | 4 | 1 | 5 |
| 13 | 5 | 1 | 1 | 5 | 5 | 1 | 5 |
| 14 | 5 | 1 | 1 | 5 | 1 | 2 | 5 |
| 15 | 5 | 1 | 1 | 5 | 1 | 3 | 5 |
| 16 | 5 | 1 | 1 | 5 | 1 | 4 | 5 |
| 17 | 5 | 1 | 1 | 5 | 1 | 5 | 5 |
| 18 | 5 | 2 | 2 | 5 | 1 | 1 | 5 |
| 19 | 5 | 3 | 3 | 5 | 1 | 1 | 5 |
| 20 | 5 | 4 | 4 | 5 | 1 | 1 | 5 |
| 21 | 5 | 5 | 5 | 5 | 1 | 1 | 5 |
| 22 | 5 | 2 | 1 | 5 | 2 | 1 | 5 |
| 23 | 5 | 3 | 1 | 5 | 3 | 1 | 5 |
| 24 | 5 | 4 | 1 | 5 | 4 | 1 | 5 |
| 25 | 5 | 2 | 1 | 5 | 2 | 1 | 5 |
| 26 | 5 | 2 | 1 | 5 | 1 | 2 | 5 |
| 27 | 5 | 3 | 1 | 5 | 1 | 3 | 5 |
| 28 | 5 | 4 | 1 | 5 | 1 | 4 | 5 |
| 29 | 5 | 5 | 1 | 5 | 1 | 5 | 5 |
| 30 | 5 | 2 | 2 | 5 | 2 | 1 | 5 |
| 27 | 5 | 3 | 3 | 5 | 3 | 1 | 5 |
| 28 | 5 | 4 | 4 | 5 | 4 | 1 | 5 |
| 29 | 5 | 5 | 5 | 5 | 5 | 1 | 5 |
| 30 | 5 | 2 | 2 | 5 | 2 | 5 | 5 |
| 31 | 5 | 3 | 3 | 5 | 3 | 10 | 5 |
| 31 | 5 | 4 | 4 | 5 | 4 | 4 | 5 |
| 33 | 5 | 5 | 5 | 5 | 5 | 5 | 5 |

TABLE 3

| Combination No. | Activity |
|---|---|
| 1 | ++++ |
| 2 | ++++ |
| 3 | ++++ |
| 4 | ++++ |
| 5 | ++++ |
| 6 | ++++ |
| 7 | ++++ |
| 8 | ++++ |
| 9 | ++++ |
| 10 | ++++ |
| 11 | ++++ |
| 12 | +++++ |
| 13 | ++++ |
| 14 | ++++ |
| 15 | +++++ |
| 16 | ++++ |
| 17 | +++++ |
| 18 | ++++ |
| 19 | ++++ |
| 20 | ++++ |
| 21 | ++++ |
| 22 | ++++ |
| 23 | ++++++ |
| 24 | ++++ |
| 25 | ++++ |
| 26 | ++++ |
| 27 | +++++ |
| 28 | ++++ |
| 29 | ++++ |
| 30 | ++++ |
| 31 | ++++ |
| 32 | ++++ |
| 33 | ++++ |

Antibacterial Activity of Natural composite preservatives
+: halo diameter not more than 3 mm
++: halo diameter from 4 to 10 mm
+++: halo diameter from 11 to 13 mm
++++: halo diameter from 14 to 16 mm
+++++: halo diameter from 17 to 19 mm
++++++: halo diameter not less than 20 mm

The concentration ratio with the highest synergistic effect (combination 23) was quantitatively and qualitatively analyzed for active ingredients, and the results are summarized in Table 4, below.

The extract from Citrus grandis contained the propenal derivative 2-methoxycinnamaldehyde as a main active ingredient. The best antibacterial activity was elicited when this active ingredient was present in an amount of 0.2%. In the extract from Matricaria recutita, apigenin and alpha-bisabolol were detected as main ingredients. The highest synergistic effect was observed when alpha-bisabolol was contained in a concentration of 0.2%. Catechins were predominantly found in the extract from Camellia sinensis. A concentration of 0.2% of EGCG (epigallocatechin gallate) was found to contribute to the best effect. Magnolol, a main ingredient found in Magnolia biondii, also contributed to the best effect at a concentration of about 0.2%. There were two main ingredients, caffeic acid and quercetin, in the extract from propolis. The best effect was detected when caffeic acid was included at a concentration of 0.3%. The best effect was elicited when alpha-thujaplicin, a main ingredient of the extract from Thujopsis dolabrata, and salicin, a main ingredient of the extract from Salix alba, were contained in concentrations of 0.2% and 0.01%, respectively.

TABLE 4
Optimal Compositions of Plant Extracts in Natural composite
preservatives

| | | Contents |
|---|---|---|
| Extracts from | Main Ingredients | (%) |
| Citrus grandis | 2-methoxycinnamaldehyde | 0.2 |
| Matricaria recutita | Alpha-Bisabolol | 0.2 |
| Camellia sinensis | EGCG | 0.2 |
| Magnolia biondii | magnolol | 0.2 |
| propolis | Caffeic acid | 0.3 |
| Thujopsis dolabrata | alpha-thujaplicin | 0.2 |
| Salix alba | Salicin | 0.01 |
| Pure water/Solvent | | Remainder to 100 |

To cosmetics or pharmaceuticals were applied the natural composite preservatives which contained the extracts in the concentration ratios shown in Table 5, below.

TABLE 5
Concentration Ratios of Plant Extracts of Naturotics in
Cosmetics or Pharmaceuticals

| | Extracts | | | | | | |
|---|---|---|---|---|---|---|---|
| | Citrus | Matricaria | Camellia | Magnolia | | Thujopsis | Salix |
| | grandis | recutita | sinensis | biondii | Propolis | dolabrata | alba |
| Contents | 1–5 | 1–5 | 1–5 | 1–5 | 1–5 | 1–5 | 1–5 |
| (%) | | | | | | | |

### EXAMPLE 4

### Antibacterial Activity of Natural Composite Preservatives

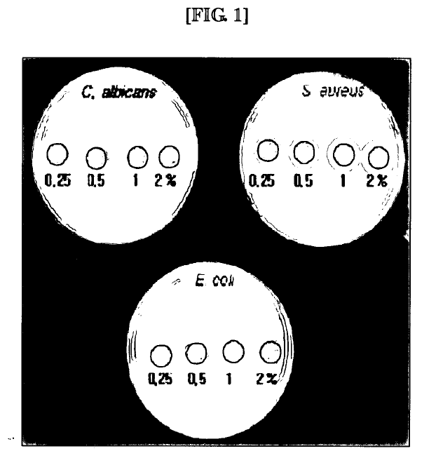

The natural composite preservatives were assayed for antibacterial activity using an agar diffusion method. Gram-positive Staphylococcus aureus, gram-negative Escherichia coli, and the fungus Candida albicans were spread over agar plates, and disks coated with concentrations of 0.25%, 0.5%, 1.0% and 2.0% of the natural composite preservatives were placed on the agar plates, before incubation for a predetermined time period. Growth inhibition halos appeared around the disks and their sizes were measured. The results are given in Table 6, below. As shown in Table 6, the natural composite preservatives of the present invention have high antibacterial activity and show better inhibition against Staphylococcus aureus and Candida albicans than E. coli. The growth inhibition halos around natural composite preservative-coated disks are shown in FIG. 1.

TABLE 6
Sizes of Inhibition Halos around Disks

| | E. coil | | | | S. aureus | | | | C. albicans | | | |
|---|---|---|---|---|---|---|---|---|---|---|---|---|
| | Concentration (%) | | | | | | | | | | | |
| | 0.25 | 0.5 | 1.0 | 2.0 | 0.25 | 0.5 | 1.0 | 2.0 | 0.25 | 0.5 | 1.0 | 2.0 |
| Halo Diameter (mm) | 10.5 | 11.5 | 13 | 16 | 14.5 | 16.5 | 18 | 20 | 13 | 15 | 17 | 19 |

### EXAMPLE 5

### Minimal Inhibitory Concentration (MIC) of Natural Composite Preservative

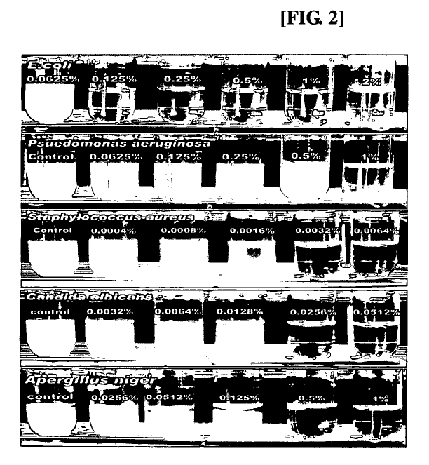

The natural composite preservatives of the present invention were measured for minimum inhibitory concentration, which is the lowest concentration of an antimicrobial that will inhibit the visible growth of a microorganism after overnight incubation. After being incubated for 24 hrs, Staphylococcus aureus, Candida albicans, Escherichia coli, Pseudomonas aeruginosa, and Aspergillus niger were respectively transferred into 3 ml of fresh media (brain heart infusion broth), along with the natural composite preservatives at predetermined concentrations. Incubation was conducted at 37° C. for 24 hrs. A sequential two-fold dilution method was used for the measurement. The results are summarized in Table 7, below. The natural composite preservative of the present invention was determined to have an MIC of 0.125% against Escherichia coli, 0.003% against Staphylococcus aureus, 0.026% against Candida albicans, 1.0% against Pseudomonas aeruginosa, and 0.5% against Aspergillus niger, with the highest inhibitory effect on gram-positive Staphylococcus aureus. The growth inhibition of microorganisms due to the natural composite preservatives is shown in FIG. 2.

TABLE 7
Minimum Inhibitory Concentration (MIC) of Naturotic

| Strains | MIC (%) |
|---|---|
| Escherichia coli | 0.125 |
| Pseudomonas aeruginosa | 1.000 |
| Staphylococcus aureus | 0.003 |
| Candida albicans | 0.026 |
| Aspergillus niger | 0.500 |

### EXAMPLE 6

### Assay of Natural composite preservatives for Antioxidant Activity

The antioxidant activity of the natural composite preservatives was analyzed with DPPH (1,1-diphenyl-2-picrylhydrazyl). DPPH is a color radical generally used in determining radical scavenging capacity. Test samples were dissolved in 4 ml of distilled water or a solvent and mixed with 1 ml of 100 μM DPPH before incubation at room temperature for 30 min. Absorbance at 517 nm was read to calculate the amount of remaining DPPH (blank: distilled water or solvent only, control: DPPH alone in distilled water or solvent). Radical Scavenging Activity (RSA) was expressed as percentages of the test groups relative to the control, as calculated by the following equation 1. The results are summarized in Table 8, below.

$$RSA ( \% ) = \frac{Abs . of hydroxylate}{Abs . of Control} \times 100 \qquad [ Equation 1 ]$$

TABLE 8
Antioxidant Effect of Natural composite preservative

| | Conc. Of | | | | | | | | | |
|---|---|---|---|---|---|---|---|---|---|---|
| | Naturotic (%) | | | | | | | | | vitamin C |
| | 0 | 0.01 | 0.05 | 0.1 | 0.2 | 0.4 | 0.6 | 0.8 | 1 | (1 mM) |
| RSA (%) | 100 | 82 | 55 | 43 | 39 | 32 | 28 | 25 | 23 | 35 |

As seen in Table 6, the natural composite preservatives according to the present invention scavenged radicals in a concentration-dependent manner.

### EXAMPLE 7

### Assay of Natural Composite Preservatives for Cytotoxicity

The cytotoxicity of the natural composite preservatives of the present invention was analyzed using an MTT assay with the human keratinocyte cell line HaCaT and normal human fibroblasts. The results are given in Table 9, below. Almost no cytotoxicity was detected at concentrations less than 7% of the natural composite preservatives, while slight cytotoxicity was observed when the natural composite preservatives were used at a concentration higher than 7%. Because the natural composite preservative is used in practice at a concentration less than 7% in cosmetics and pharmaceuticals, it is safe for the body.

TABLE 9
Cytotoxicity of the Natural composite preservative

| | Naturotics Conc.(%) | | | | | | | |
|---|---|---|---|---|---|---|---|---|
| | 0 | 0.1 | 0.5 | 1 | 3 | 5 | 7 | 10 |
| Cell Viability | 100 | 102 | 99.8 | 99.7 | 99.5 | 99.5 | 97.2 | 85.6 |

### EXAMPLE 8

### Preservative Effect of the Natural Composite Preservatives on Cosmetics

In order to analyze the preservative effect of the natural composite preservatives prepared in the preparation example on cosmetics, lotions and skin toners, both containing the natural composite preservatives at a concentration of 2%, were prepared as follows.

A. Lotion Containing the Natural Composite Preservative

A lotion containing the natural composite preservative was prepared from the composition given in Table 10 using a conventional method.

TABLE 10

| Ingredients | Contents |
|---|---|
| Naturotics | 2.00 |
| Cetearyl-6 olivate | 2.00 |
| arachidyl alcohol/behenyl alcohol/ | 0.90 |
| arachidyl glucoside | |
| tocopheryl acetate | 0.05 |
| neopentylglycoldiheptanoate | 2.00 |
| polydecene | 2.50 |
| stearic acid | 0.60 |
| Dimethicone | 0.40 |
| triethanol amine | 0.10 |
| Disodium EDTA | 0.30 |
| carbomer | 0.40 |
| xanthan gum | 0.04 |
| perfume | Trace |
| purified water | Remainder to |
| | form 100% |
| Total | 100 |

B. Lotion Lacking the Natural Composite Preservative (Control)

A lotion was prepared from the composition given in Table 11, which is the same as in section A, with the exception that purified water was used instead of the natural composite preservative.

TABLE 11

| Ingredients | Contents |
|---|---|
| Cetearyl-6 olivate | 2.00 |
| arachidyl alcohol/behenyl alcohol/ | 0.90 |
| arachidyl glucoside | |
| tocopheryl acetate | 0.05 |
| neopentylglycoldiheptanoate | 2.00 |
| polydecene | 2.50 |
| stearic acid | 0.60 |
| dimethicone | 0.40 |
| triethanol amine | 0.10 |
| Disodium EDTA | 0.30 |
| Carbomer | 0.40 |
| xanthan gum | 0.04 |
| Perfume | Trace |
| purified water | Remainder to |
| | form 100% |
| Total | 100 |

C. Skin Toner Containing the Natural Composite Preservative

A skin toner containing the natural composite preservative was prepared from the composition given in Table 12 using a conventional method.

TABLE 12

| Ingredients | Contents |
|---|---|
| Naturotics | 2.00 |
| Propylene glycol | 3.50 |
| Phenyltrimethicone | 0.70 |
| PPG-26-Buteth-26/ | 0.40 |
| PEG-40 hydrogenated castor oil | |
| PEG-8 dimethicone | 1.00 |
| Ethanol | 5.00 |
| Disodium EDTA | 0.10 |
| Butylene glycol | 2.50 |
| triethanol amine | 0.15 |
| Perfume | Trace |
| purified water | Remainder to |
| | form 100% |
| Total | 100 |

D. Skin Toner Lacking the Natural Composite Preservative (Control)

A skin toner was prepared from the composition given in Table 13, which is the same as in section C, with the exception that purified water was used instead of the natural composite preservative.

TABLE 13

| Ingredients | Contents |
|---|---|
| Propylene glycol | 3.50 |
| Phenyltrimethicone | 0.70 |
| PPG-26-Buteth-26/ | 0.40 |
| PEG-40 Hydrogenated castor oil | |
| PEG-8 dimethicone | 1.00 |
| Ethanol | 5.00 |
| Disodium EDTA | 0.10 |
| Buthylene glycol | 2.50 |
| triethanol amine | 0.15 |
| Perfume | Trace |
| purified water | Remainder to |
| | form 100% |
| Total | 100 |

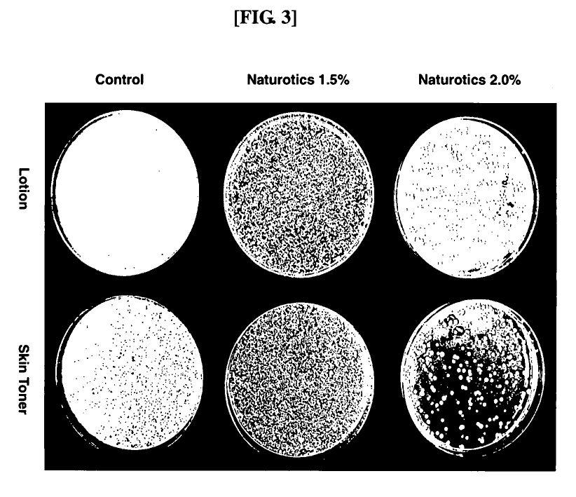

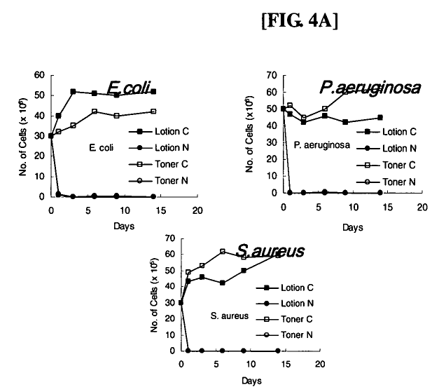

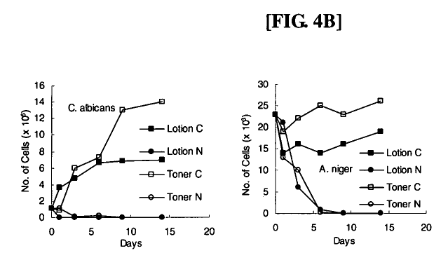

The lotions prepared in section A (containing 2% of the natural composite preservative), section B (lacking the natural composite preservative) and skin toners prepared in section C (containing 2% of the natural composite preservative) and D (lacking the natural composite preservative) were assayed for preservation according to the Microbial Challenge Test proposed by the European Pharmacopoeia Commission (E.P.) in 1996 (Letters in Applied Microbiology 2002, 35, 385-389). This test was carried out on Escherichia coli, Pseudomonas aeruginosa, Staphylococcus aureus, Candida albicans, and Aspergillus niger. The preserved states of the lotions and skin toners were measured four times at regular intervals of 5 days for 20 days. As shown in the lotions and skin toners, each containing 1.5 and 2% of the natural composite preservative of FIG. 3, the number of E. coli in both the lotions and the skin toners was significantly decreased in a manner dependent on the dose of the natural composite preservative over three days. In addition, almost all microorganisms in the lotion and the skin toner, both containing 2% of the natural composite preservative, died within five days while they grew in abundance for 20 days in the lotion and skin toner lacking the natural composite preservative (FIGS. 4a and 4b), which demonstrates that the natural composite preservative of the present invention has potent preservative activity.

### EXAMPLE 9

### Safety of the Natural Composite Preservatives for Human Skin

The natural composite preservatives of the present invention, identified as having high in antibacterial and preservative activity, were analyzed for safety on the skin according to a dermal cumulative irritation test method.

Squalene-based formulations containing 0.1%, 1%, 5%, and 10% of the natural composite preservatives were applied to the forearm of 30 healthy adults through patches for 24 hrs every other day, and the patch was changed with a fresh one 9 times in total since the first application in order to examine whether the natural composite preservatives irritated the skin or not.

The patch test was performed using a Finn chamber (Epitest Ltd, Finland). The topical agents were loaded dropwise in an amount of 15 μl per patch on the chamber. Every round of the patch application, the response of the skin was scored using the following equation 2, and the results are given in Table 14, below.

$$Avg . Response Degree = \frac{Response Index \times Response Degree}{Total No . of subjects \times highest score ( 4 points )} \times \frac{100}{No . of Test ( 9 rounds )} \qquad [ Equation 2 ]$$

In regard to the response degree, 1 point was provided for ±, 2 points for +, 4 points for ++. When the average response degree was less than 3, the composition was determined to be safe for the skin.

TABLE 14

| | Nos. of Subjects in response | | | | | | | | | |
|---|---|---|---|---|---|---|---|---|---|---|
| | Week 1 | | | Week 2 | | | Week 3 | | | Avg. |
| | 1^{st} | 2^{nd} | 3^{rd} | 4^{th} | 5^{th} | 6^{th} | 7^{th} | 8^{th} | 9^{th} | Response |
| Test Material | ±+⁺⁺ | ±+⁺⁺ | ±+⁺⁺ | ±+⁺⁺ | ±+⁺⁺ | ± +⁺⁺ | ±+⁺⁺ | ++⁺⁺ | ±+⁺⁺ | Degree |
| Squalene | 2 | — | — | — | — | — | — | — | — | 0.18 |
| (Control) | | | | | | | | | | |
| Naturotics | 2 | 0 | — | 0 | | | | | | 0.18 |
| (0.1%, Group 1) | | | | | | | | | | |
| Naturotics | 3 | 1 | | 1 | | | | | | 0.37 |
| (0.5%, Group 2) | | | | | | | | | | |
| Naturotics | 2 | 0 | | 0 | | | | | | 0.18 |
| (1%, Group 3) | | | | | | | | | | |
| Naturotics | 3 | 1 | | 1 | | | | | | 0.35 |
| (10%, Group 4) | | | | | | | | | | |
| No. of subjects | 30 | 30 | 30 | 30 | 30 | 30 | 30 | 30 | 30 | |

As shown in Table 14, the subjects corresponding to ±, + and ++ in Test Group 1 numbered 2, 0 and 0, respectively, while the others showed no response. According to equation 2, The average response degrees of Test Groups 1 to 4 were calculated to be 0.18, 0.37, 0.18 and 0.37, respectively, which all are less than 3, demonstrating that the natural composite preservative of the present invention causes no noticeable cumulative irritation and is safe for human skin.

### EXAMPLE 10

### Acute Oral Toxicity Assay

In order to examine whether the natural composite preservative of the present invention is safe for application to food, an acute oral toxicity assay was conducted thereon. 20 SPF SD rats, all 5˜6 weeks old, were orally administered with the natural composite preservatives of the present invention under the following conditions.
Temperature and humidity: 22±2° C., RH 50±10%
Light-dark cycle: fluorescent lamp (Turn-On at 8 A.M., Turn-Off at 8 P.M.)
Illuminance: 200˜300 Lux
allowed freely to approach UV-treated water
Dilutions of the test substance in sterile distilled water were administered while the same volume of sterile distilled water was used as a control.

On the day of administration, the rats were observed for general conditions every hour for 4 hrs after the administration. From 1 to 14 days after the administration, the rats were carefully observed once a day for general state, toxic symptoms, motility, appearance, change in the autonomic nervous system, and death. Autopsies were also performed to determine the toxicity of the natural composite preservative. The natural composite preservative of the present invention was determined to have an LD₅₀ of 24,500 mg/kg B.W. and no toxicity.

As described hitherto, the natural composite preservative of the present invention has potent antibacterial activity against a wide spectrum of microorganisms. Furthermore, the natural composite preservatives of the present invention are found to be safe for the body, with the production of neither toxicity nor irritation, the result of various safety tests. Therefore, the natural composite preservatives of the present invention are very useful in the preservation of cosmetics and pharmaceuticals.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A natural composite preservative, comprising a combination of extracts from Camellia sinensis, Matricaria recutita, Citrus grandis, Magnolia biondii, propolis, Thujopsis dolabrata and Salix alba.

2. The natural composite preservative according to claim 1, wherein the combination contains extracts from Citrus grandis, Camellia sinensis, propolis, Magnolia biondii, Matricaria recutita, Salix alba, and Thujopsis dolabrata in a concentration ratio of 5:1˜10:1:5:1˜10:1:5 (Citrus grandis:Camellia sinensis:propolis:Magnolia biondii:Matricaria recutita:Salix alba:Thujopsis dolabrata).

3. The natural composite preservative according to claim 1, wherein the combination comprises epigallocatechin gallate (EGCG), alpha-bisabolol, 2-methoxycinnamaldehyde, magnolol, caffeic acid, alpha-thujaplicin and salicin.

4. The natural composite preservative according to claim 3, wherein EGCG, bisabolol, 2-methoxycinnamaldehyde, magnolol, caffeic acid, alpha-thujaplicin and salicin are contained in a concentration ratio of 0.01˜5.0%.

5. A product, comprising the natural composite preservative of one of claims 1 to 4.

6. The product according to claim 5, wherein the product is a cosmetic.

7. The product according to claim 5, wherein the product is a pharmaceutical.

8. The product according to claim 5, comprising the natural composite preservative in an amount of 0.001˜30% by weight based on total weight of the product.
